(19) United States
(12) Patent Application Publication
(10) Pub. No.: US 2022/0395673 A1
(43) Pub. Date: Dec. 15, 2022

(54) DRUG ELUTING GUIDE WIRE

(71) Applicants: Pawel Wisniewski, Wroclaw (PL); WISMED PL SP Z.O.0. A COMPANY INCORPORATED IN POLAND; NUMBER KRS 0000751368, Wroclaw (PL)

(72) Inventor: Pawel Wisniewski, Wroclaw (PL)

(21) Appl. No.: 17/621,565

(22) PCT Filed: Jun. 21, 2020
(86) PCT No.: PCT/ZA2020/050034

§ 371 Date: Dec. 21, 2021

(30) Foreign Application Priority Data
Jun. 21, 2019 (ZA) 2019/02878

(51) Int. Cl. A61M 25/09; A61K 31/137

(52) U.S. Cl. CPC A61M 25/09; A61K 31/137; A61M 2025/09133; A61M 2205/0222; A61M 2210/1078

(57) ABSTRACT

A drug eluting guide wire comprises a body aperture having low friction characteristics. The guide wire includes an effective leading end segment that is formed along its outer surface with a rapid action body aperture drug eluting agent serving as a vasilodating substance. The low friction characteristics are achieved by the leading end segment of the wire being covered with a low friction lubricating substance such as hydrogel that is loaded with a drug eluting substance in the form of isoprenaline in constituting the segment. Isoprenaline serves a constricted body aperture rapid action relaxing function in amongst others, or at least the case of the guide wire, is used to access a kidney along a ureter in response to temporarily paralyzing the smooth muscles of the ureteric wall resulting in widening of the lumen of the ureter.

### (2) FIELD OF THE INVENTION

This invention relates to drug eluting guide wire for use in relaxing body tissue by way of a vasodilating substance during use of such guide wire under body procedures involving guide wire application. While not so limited the invention find useful application in the field of uretetic orifice if not full viscus relaxation in the process kidney access via a ureter.

### (3) BACKGROUND TO THE INVENTION

Nephrolithiasis (kidney stones) is one of the most common urological condition in modern times, affecting one in ten people. During urological procedures to secure access to a kidney in order to visualize its interior and/or to fragment and remove kidney stones or perform other procedures it is necessary to introduce instruments from the bladder end of the ureter up to the kidney. The instruments need to be advanced through the ureter from its narrower end up to the wider end that is proximal to and joining the kidney to the bladder. The ureter is narrower than such instruments with its lumen normally closed remote from the bladder while only opening sporadically. It is at least one object of this invention to facilitate kidney access by promoting ureter relaxation during the insertion of a guide wire conventionally used as railroading support for other instruments in the performance of kidney associated techniques.

### (4) DESCRIPTION OF PRIOR ART

Several techniques to widen a ureter prior to instrument insertion are known in the art. One technique involves the use of mechanical force to dilate a ureter that is however not without risk of trauma as force is used to stretch such ureter. In another technique a vasoactive substance such as isoprenaline is injected to temporarily paralyze the smooth muscles of the ureteric wall thus causing it to widen. This technique is seldomly practiced in being very cumbersome. Under the Seldinger technique a very thin stent fitted over a guide wire is inserted along a ureter and then left so positioned for a few days. The presence of such foreign body paralyses smooth muscles and dilates the ureter enough to allow insertion of a scope, sheath or other equipment. The drawback is that a patient requires at least two procedures several days apart. While the prior art is referenced in the use of guide wires for kidney procedures it will appreciated that its use not necessarily so limited.

### (5) BRIEF DESCRIPTION OF THE DRAWING

The invention is now described, by way of example, with reference to the accompanying drawings. In the drawings

FIG. 1 shows a drug eluting guide wire in the form of a kidney access eluting guide wire involving a conventional body aperture if not full body viscus guide wire as supplemented into forming the guide wire of the invention in three dimensional partly cut out view,

FIG. 2 shows the drug eluting guide wire in diagrammatic side elevation, and

FIG. 3 shows a variety of conventional guide wire leading end shapes.

### (6) DETAILED DESCRIPTION OF THE DRAWINGS

In referring to the drawings a drug eluting guide wire including a conventional low friction characteristics constricted body aperture if not full body viscus access guide wire 10 is generally indicated by reference numeral 12.

The guide wire 12 is constituted by being formed along a leading end segment 14 of its outer surface with rapid action body aperture drug eluting agent serving as vasilodating substance, as discussed in more detail below.

Guide wires 10 are conventionally constituted with stiff though yieldable shaft sections 16 carrying soft hydrophilic leading end tips 18 typically of a length of about 5 cm. The low friction characteristics of conventional guide wires 10 in being required to penetrate sensitive body apertures such as uretetic orifices if not full viscii such as ureters are conventionally provided by low friction lubricating substances covering at least part of their lengths as from their leading ends 20. Lubricious hydrogel is typical of such substances.

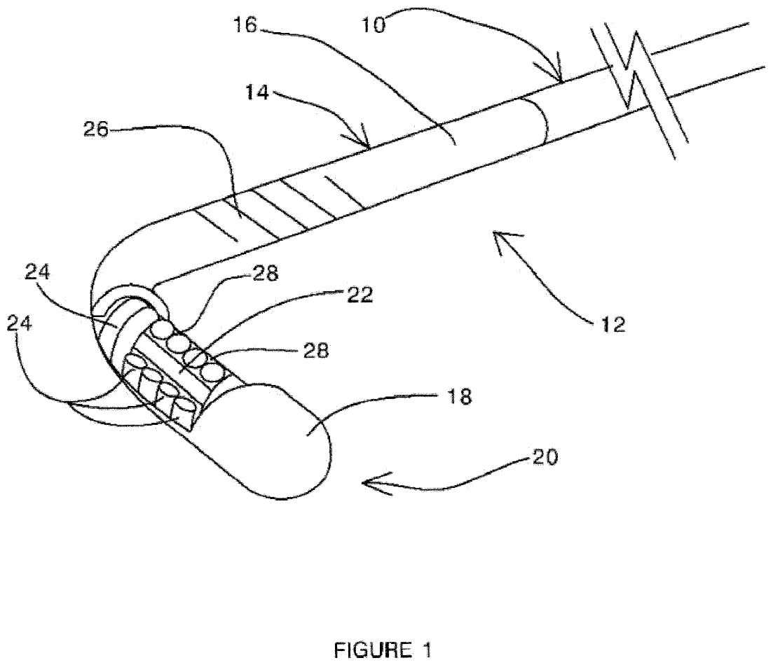

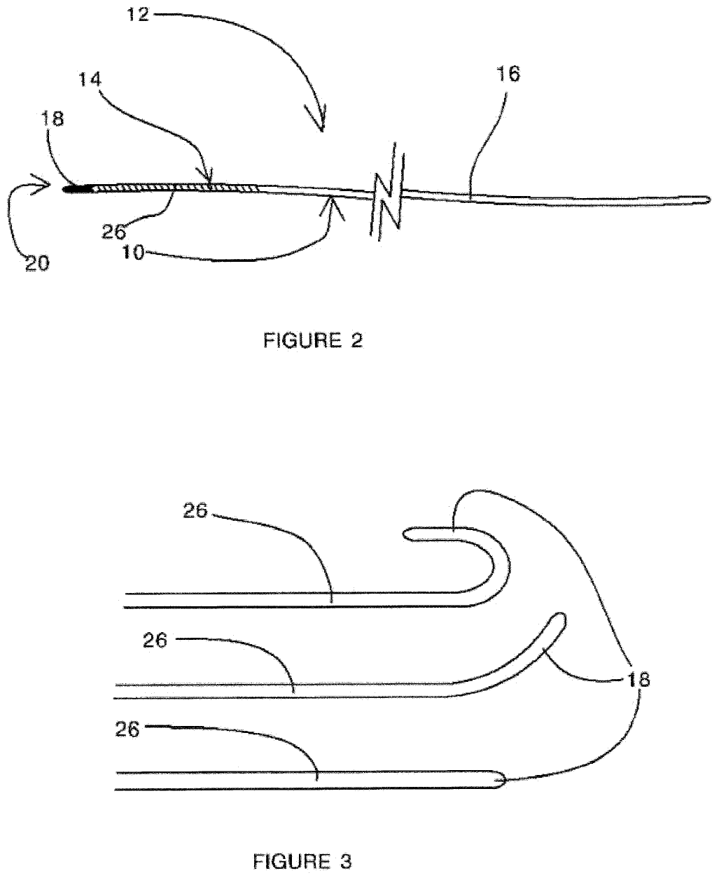

In more specifically referring to FIG. 1, guide wires 10 conventionally have Nitinol cores 22 to retain shape memory and spiral reinforcement 24 to prevent kinking. The reinforcement can be in form of flat or round wire spring. Flat wire reinforcement offers more rigidity with Teflon being painted there over after contraction. In the case of round wire reinforcement and more specially referring to FIG. 4, the reinforcement is first covered by Teflon coating and then wound around the core 22. This has the effect of such round wire configuration having good low friction characteristics once covered with hydrogel 26 as it permits the application of a larger extent of hydrogel coverage while resulting in smaller contact area between the guide wire 10 and a body aperture. With round wire reinforcement the hydrogel is also protectively located in the grooves 28 along such reinforcement before wiping during surgery.

The characteristics of the hydrogel coating can be enhanced by basing it on chitosan that has antibacterial properties. Its stability can be improved by the introduction of an intermediate layer of polydopamine between the surface of the guide wire 10 and the hydrogel coating.

However arranged, urological conventional guide wires 10 typically have outside diameters of in the order of 0.45-1.00 mm.

In whichever way a guide wire 10 is conventionally constituted, the guide wire 12 of the invention thus incorporates drug eluting substance loaded to the hydrogel 26 as covering a conventional guide wire 10 in forming the drug eluting agent carrying wire section 14. The drug eluting substance can for example be incorporated into the hydrogel by being diffused into its pores. Alternatively, the drug eluting substance can be incorporated into the hydrogel by being incorporated there into during the process of hydrogel formation. In a further embodiment the drug eluting substance can be incorporated into the hydrogel by the pre-formulation of agent nanoparticles in an oil/water system followed by its subsequent swelling into the pores of the hydrogel. The amount and the kinetics of agent release from the coating is naturally a function of the amount of coating applied, the amount of substance loaded, the molecular weight of the substrates and the degree of crosslinking of the hydrogel.

While the guide wire 12 can be used in a variety of body applications its application in kidney related procedures is of particular importance to this invention. The drug eluting substance is in such case in the form of isoprenaline that, as loaded to the hydrogel, covers the conventional guide wire 10 in forming the isoprenaline incorporating drug eluting agent carrying section 14 of in the order of 30 cm in defining the drug eluting guide wire 12.

In use the guide wire 12 is conventionally passed along a ureter causing the isoprenaline to become released invasilodating at least the ureteric orifice if not the full extent of the ureter during and subsequent to such action. This is achieved by the isoprenaline in a controlled way though quite rapidly relaxing and thus temporarily paralyzing the smooth muscles of the ureteric wall resulting in widening of the lumen of the ureter while also causing it to stop its peristaltic activity. As said above the agent release effect is dependent on a variety of circumstances.

Once the ureter is properly relaxed other kidney application instruments are railroaded there over to perform the relevant kidney associated procedure(s).

While the equipment of the invention and its technique of use are described with its specific application in relaxing a ureter it will be appreciated that its scope is not so limited.

It is an advantage of this invention at least as specifically described that it provides an atraumatic, safe and non-cumbersome method of widening the lumen of the ureter and relaxing its walls prior to the insertion (railroading) of other equipment such as a scope or sheath during or subsequent to the process of guide wire insertion in the performance of kidney associated techniques such as the breaking up of kidney stones. An additional advantage of the temporary paralysis of a uretetic orifice if not the full ureter in using the guide wire of the invention is the achievement of better drainage of irrigation fluid from the kidney and the lowering of dangerous intrarenal pressures. When used in conjunction with railroading the guide wire of the invention causes a reduction of ureter trauma associated with such railroading.

1. A drug eluting guide wire comprising:
a body aperture having low friction characteristics,
wherein an effective leading end segment of the guide wire is formed along its outer surface with a rapid action drug eluting agent serving as a vasilodating substance in causing a relaxation effect during exposure to at least the body aperture for the bodily positioning of the guide wire along a body viscus in promoting access to the interior of a body location there beyond.

2. A drug eluting guide wire as claimed in claim 1, in which the low friction characteristics are constituted with a stiff though yieldable shaft section carrying a soft hydrophilic leading end tip.

3. A drug eluting guide wire as claimed in claim 1, in which at least part of the body aperture is caused to have low friction characteristics by its being covered along at least part of its length extending from its leading end with low friction lubricating substance for promoting passage along a constricted body aperture cum viscus with at least part of the length of lubricating substance coverage, as extending from the leading end of the guide wire, is loaded with drug eluting substance in forming the effective drug eluting agent carrying leading end segment.

4. A drug eluting guide wire as claimed in claim 3, in which the low friction lubricating substance is in the form of a lubricious hydrogel.

5. A drug eluting guide wire as claimed in claim 4, in which the drug eluting substance is incorporated into the hydrogel by being diffused into its pores.

6. A drug eluting guide wire as claimed in claim 4, in which the drug eluting substance is incorporated into the hydrogel by being incorporated there into during the process of hydrogel formation.

7. A drug eluting guide wire as claimed in claim 4, in which the drug eluting substance is incorporated into the hydrogel by the pre-formulation of substance nanoparticles in an oil/water system followed by its subsequent swelling into the pores of the hydrogel.

8. A drug eluting guide wire as claimed in claim 4, in which at least in the case of its being used for accessing a kidney along a ureter as viscus the drug eluting substance is in the form of isoprenaline.

9. A drug eluting guide wire as claimed in claim 8, in which the length of the effective leading end segment as thus loaded with the isoprenaline is on the order of 30 centimeters (“cm”).

10. A drug eluting guide wire as claimed in claim 1, wherein the guide wire is configured for full body viscus access.

11. A drug eluting guide wire as claimed in claim 11, wherein the relaxation effect occurs during full body viscus access of guide wire.
